(19) United States
(12) Patent Application Publication
(10) Pub. No.: US 2023/0083184 A1
(43) Pub. Date: Mar. 16, 2023

(54) OPTIMIZATION OF PROCESSES FOR MANUFACTURING AMORPHOUS SILICON

(71) Applicant: International Business Machines Corporation, Armonk, NY (US)

(72) Inventors: FAUSTO MARTELLI, Stockton Heath (GB); MALGORZATA JADWIGA ZIMON, Warrington (GB)

(21) Appl. No.: 17/471,266

(22) Filed: Sep. 10, 2021

(51) Int. Cl. G06F 30/25

(52) U.S. Cl. CPC G06F 30/25; G06F 2111/10

(57) ABSTRACT

Optimizing a processes for the manufacture of amorphous silicon by generating molecular dynamics simulations of amorphous silicon across a range of manufacturing conditions, applying a local order metric algorithm to each of the simulations to detect formation of crystalline structures within each of the simulations based on a local order metric determined by the algorithm, determining material quality for each simulation as a function of the local order metric and the detected crystalline structures, modifying manufacturing conditions according to a desired material quality and applying the modified manufacturing conditions to the manufacturing process of amorphous silicon. A molecular dynamics simulation of amorphous silicon may be generated using modified manufacturing conditions, applying the local order metric to the modified simulation to detect whether the crystalline structures are formed, if the crystalline structures are formed, repeating the modifying and dynamics simulation steps until no crystalline structures are detected.

### BACKGROUND

This disclosure is directed to manufacture of amorphous silicon, and more particularly to methods and systems for optimizing the manufacturing process to improve the quality of amorphous silicon.

Amorphous materials are like crystals in the way that atomic motion is strongly suppressed and like liquids in that atoms are arranged in a disordered configuration. Amorphous silicon (a-Si) is a non-crystalline solid form of silicon typically used for large area electronics, with major applications in displays, image sensing, solar power conversion (photovoltaics) thin-film transistors and electrodes in batteries. There are numerous procedures that lead to the formation of a-Si and the manufacturing protocols are often provided by atomic-based molecular dynamics (MD) simulations, which allow to explore a wide range of possible routes to produce the most cost-effective conditions.

Very often the amorphous matrix embeds traces, also known as grains, of the crystal structure that are small enough to be undetected by experimental techniques. Such grains represent a problem for the manufacturing industry because amorphous states are always unstable and over time they transform into the crystal form. Grains embedded in the amorphous matrix speed the transition to crystal, hence affecting its properties and shortening the lifetime of the material. Therefore, it is challenging to precisely estimate the quality of the end material during the manufacturing process and predict its efficiency over time.

### SUMMARY

In one embodiment, a method for optimizing processes for the manufacture of amorphous silicon is disclosed. In some embodiments, processes for manufacturing amorphous silicon are optimized to achieve a desired quality. In one embodiment, a local order metric (LOM) is applied to the molecular dynamics simulations for estimating order in the amorphous silicon molecular system to identify the formation of crystallites under given conditions.

One embodiment of a computer implemented method for optimizing a process for the manufacture of amorphous silicon includes the steps of generating molecular dynamics simulations of amorphous silicon across a range of manufacturing conditions, applying a local order metric algorithm to each of the simulations to detect formation of crystalline structures within each of the simulations based on a local order metric determined by the algorithm, determining material quality for each simulation as a function of the local order metric and the detected crystalline structures, modifying manufacturing conditions according to a desired material quality and applying the modified manufacturing conditions to the manufacturing process of amorphous silicon. The method may also include generating a modified molecular dynamics simulation of amorphous silicon using the modified manufacturing conditions, applying the local order metric to the modified simulation to detect whether the crystalline structures are formed, if the crystalline structures are formed, repeating the modifying and dynamics simulation steps until no crystalline structures are detected.

A computer system that includes one or more processors operable to perform one or more methods described herein also may be provided.

A computer readable storage medium storing a program of instructions executable by a machine to perform one or more methods described herein also may be provided.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 is a block diagram of one embodiment of the system disclosed in this specification.

FIG. 2 is a diagram depicting the application of the LOM algorithm to detect crystalline structures.

FIG. 3 is a diagram depicting the application of the LOM algorithm to detect crystalline structures.

FIG. 4 is a diagram depicting the application of the LOM algorithm to optimize the manufacturing protocol.

FIG. 5 is a flow diagram of one embodiment of the method disclosed in this specification.

FIG. 6 is a flow diagram of one embodiment of the method disclosed in this specification.

FIG. 7 is a block diagram of an exemplary computing system suitable for implementation of the embodiments of the invention disclosed in this specification.

### DETAILED DESCRIPTION

In one embodiment, a method for optimizing processes for the manufacture of amorphous silicon is disclosed. In one embodiment, processes for manufacturing amorphous silicon are optimized to achieve an improved quality. In some embodiments, processes for manufacturing amorphous silicon are optimized to achieve a desired quality. In one embodiment, a local order metric (LOM) is applied to molecular dynamics simulations for estimating order in the amorphous silicon molecular system to identify the formation of crystallites under given conditions. The combination of a molecular dynamics simulations with the LOM enables an efficient and more accurate estimation of the quality of the amorphous silicon that can presently be achieved by prior art simulations. The combination of a molecular dynamics simulations with the LOM solves a long-lasting technological problem in the manufacture of amorphous silicon by specifically identifying manufacturing conditions that may be the cause defects and optimizing those conditions according to a desired material quality, resulting in improved materials, such as for solar cell applications.

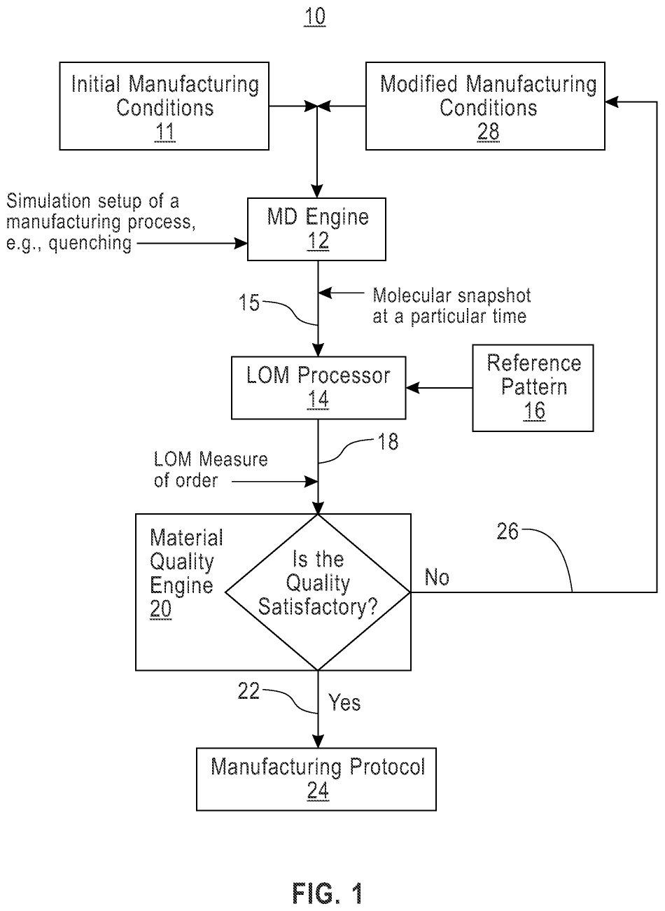

FIG. 1 is one embodiment of a system for optimizing a process for the manufacture of amorphous silicon. The system 10 includes a molecular dynamics (MD) engine 12 that generates simulations of crystalline silicon under a range of different manufacturing conditions.

Molecular dynamics (MD) simulation is a technique by which one generates the atomic trajectories of a system of N particles by numerical integration of Newton's equation of motion, for a specific interatomic potential, with certain initial conditions and boundary conditions. The MD computer simulations provide an understanding of the properties of the assemblies of molecules in terms of their structure and the microscopic interactions between them. The MD simulation provides information on structural and dynamical properties of the system such as transport coefficients, time-dependent responses to perturbations, rheological properties and spectra. The atomic-scale structure of amorphous silicon is traditionally approximated with all atoms in locally “crystal-like”, i.e., distorted, tetrahedral environments.

In one embodiment, an MD simulation of quenching during the amorphous silicon manufacturing process is generated. Simulating quenching from the melt is one technique for generating amorphous model networks. In this technique, one starts with a liquid and progressively lowers the temperature, “freezing in” an amorphous structure. During quenching, silicon changes from a high-coordination metallic liquid to a tetrahedral-like amorphous state. In one embodiment, a plurality of quench simulations can be performed in which the quench rate, and thus the run-time, is varied by several orders of magnitude. The simulations may be performed using a variable-volume and a constant-pressure.

The results 15 of the plurality of molecular simulations generated by MD engine 12 using initial manufacturing conditions 11 are input to a local order metric (LOM) processor 14. The results 15 can be data representing a molecular snapshot at one or more particular points in time during the formation of amorphous silicon. The LOM processor 14 applies a local order metric algorithm to the simulations to detect crystalline structures during the development of the simulated amorphous silicon. In one embodiment, a reference pattern 16 is input to the LOM process 14 to identify the molecular order based on shape matching. Experiments and computer simulations indicate that the local structure of amorphous silicon is tetrahedral, i.e., a given molecule is located at the center of a tetrahedron and its four nearest-neighbors are roughly located at the corner of such a tetrahedron. Therefore, in one embodiment, in order to probe the short-range order of amorphous silicon, LOM processor 14 uses a regular tetrahedron as the reference structure 16.

In one embodiment, data representing the features of a tetrahedron or unit cell (basic building block) of crystalline silicon is used as the reference pattern 16. The MD simulation data and the reference pattern feature data are used by the LOM algorithm to calculate a local order metric. In one embodiment, the procedure is independently performed for every atom in the system. In one embodiment, the computational effort can be distributed among processes based on a number of particles.

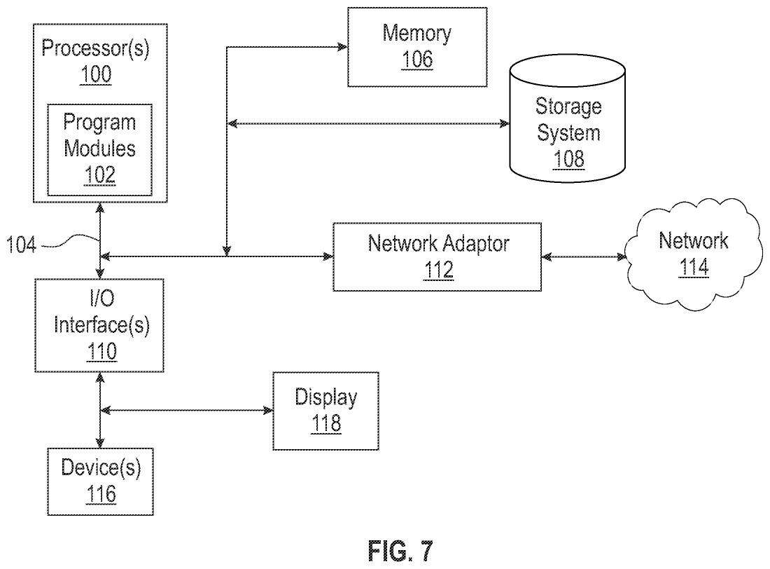

The resultant LOM 18, which is a measure of the crystalline order, is input to material quality engine 20. The material quality engine 20 estimates the material quality under the given manufacturing protocol as a function of the LOM. In one embodiment, the material quality engine 20 determines material quality for each simulation as a function of the local order metric and the detected crystalline structures. If the quality is determined to be satisfactory, by for example, meeting a desired quality measure, the manufacturing conditions 22 used in the simulation are used in the protocol 24 to manufacture amorphous silicon. If the quality is determined not to be satisfactory, by for example, not meeting a desired quality measure, the LOM and data indicating the detected crystalline structures 26 are used to generate modified manufacturing conditions 28 which are input to the MD engine. The modified manufacturing conditions 28 are run through the system again and the process is repeated until the desired quality is achieved. LOM processor 14 and material quality engine 20 may be implemented in a computer system as shown in FIG. 7 and/or a non-transitory computer readable medium to be described later.

In one embodiment, the LOM maps the local order of each atom onto a number in the range between 0 and 1. Whenever such number is greater than 0.8, it is determined that the atom is part of a small crystallite. For example, an amorphous sample should be composed by atoms with LOM<0.8 to meet a desired measure of quality for a particular application. The material quality in this example is reflected by the number of atoms having a LOM>0.8. Therefore, each crystalline structure detected has an LOM determined by the algorithm and the quality is based on the LOM and the number of crystalline structures having the metric.

The use of the LOM algorithm enables improved optimization of the manufacturing conditions. As there are numerous procedures leading to development of amorphous silicon, the identification of local patterns can be performed for all of the simulated settings in order to identify the most beneficial outcome.

The local order metric algorithm used by the LOM processor 14 measures the degree of order present in the neighborhood of an atomic or molecular site in a condensed medium, in this case amorphous silicon. The LOM algorithm is endowed with a high-resolving power and allows one to look for specific ordered domains defined by the location of selected atoms in the reference structure 16. Typically, the reference structure 16 is taken to be the local structure of a perfect crystalline phase.

In one embodiment, the LOM processor 14 probes the evolving state of the amorphous silicon molecular structure under various static and changing manufacturing conditions using the local order metric algorithm. The local environment of an atomic site j in the snapshot of a molecular dynamics simulation 15, defines a local pattern formed by M neighboring sites. Typically, these include the first and/or the second neighbors of the site j. There are N local patterns, one for each atomic site j in the system. The local reference structure 16 is the set of the same M neighboring sites in an ideal lattice of choice, the spatial scale of which is fixed by setting its nearest neighbor distance equal to d, the average equilibrium value in the system of interest, in this case amorphous silicon. For a given orientation of the reference structure 16 and a given permutation P of the indices of pattern 15, the LOM algorithm defines S(j) as the maximum overlap between pattern and reference structure in the j neighborhood by the equation (1):

$$S ( j ) = {max}_{( θ , ϕ , ψ ,} \text{?} {\prod }_{i = 1}^{M} exp - \frac{{\vert {P}^{j} \text{?} - {R}^{j} \text{?} \vert}^{2}}{2 M {σ}^{2}}$$

$$\text{?} \text{indicates text missing or illegible when filed}$$

Where θ, ϕ, and ψ are Euler angles, P [symbol] and Rₜ^{j} are the pattern and the reference position vectors in the laboratory frame of the M neighbors of site j, respectively. The index [symbol] represents the permutation of indices to compute the distance in eq.(1) and that maximizes eq.(1) for a given set of Euler angles. The parameter σ controls the spread of the Gaussian functions. The LOM algorithm (1) satisfies the inequalities 0≤. S(j)≤1. The two limits correspond, respectively, to a completely disordered local pattern (S(j)→0) and to an ordered local pattern matching perfectly the reference (S(j)→1). As mentioned above, in one embodiment, the LOM maps the local order of each atom onto a number in the range between 0 and 1. For example, whenever the number is greater than 0.8, it is determined that the atom is part of a small crystallite. The threshold 0.8 is obtained when the value of sigma in equation (1) is fixed at ¼ of the distance between nearest neighbors.

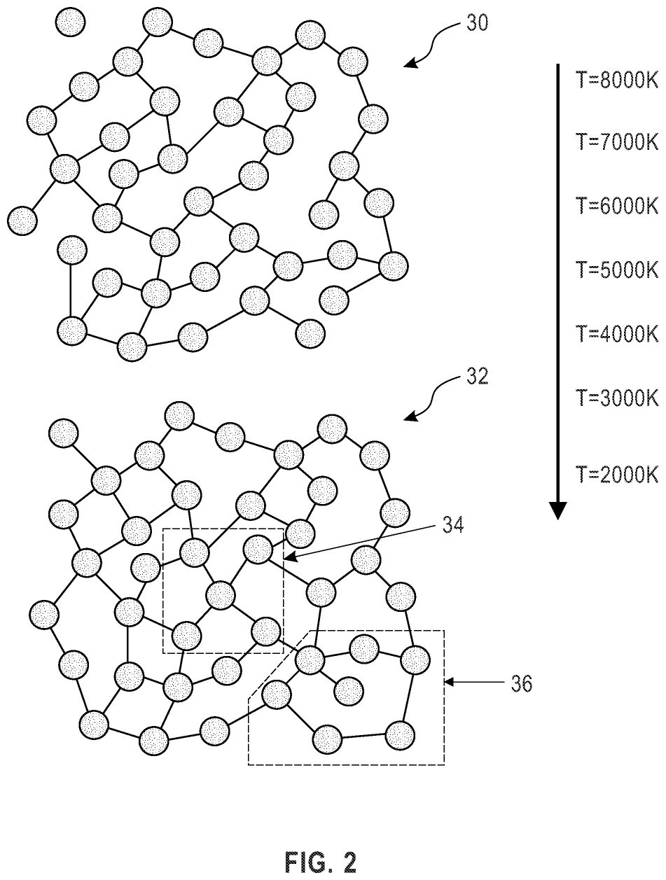

FIGS. 2-4 depict one example of how the LOM algorithm can be used to optimize a process for the manufacture of amorphous silicon to improve the quality. For a given atom j, the vector P is first computed evaluating the distances between the atom j and the set of M neighbors. This distance defines the value of the gaussian domain which can be set to ¼ of the distance. Then the vector R is computed evaluating the distance between the atom j and the set of M atoms composing the ideal reference structure. The set of M neighbors is iteratively rotated and their distance to the atom j increased or reduced to maximize equation (1) keeping the value of sigma fixed.

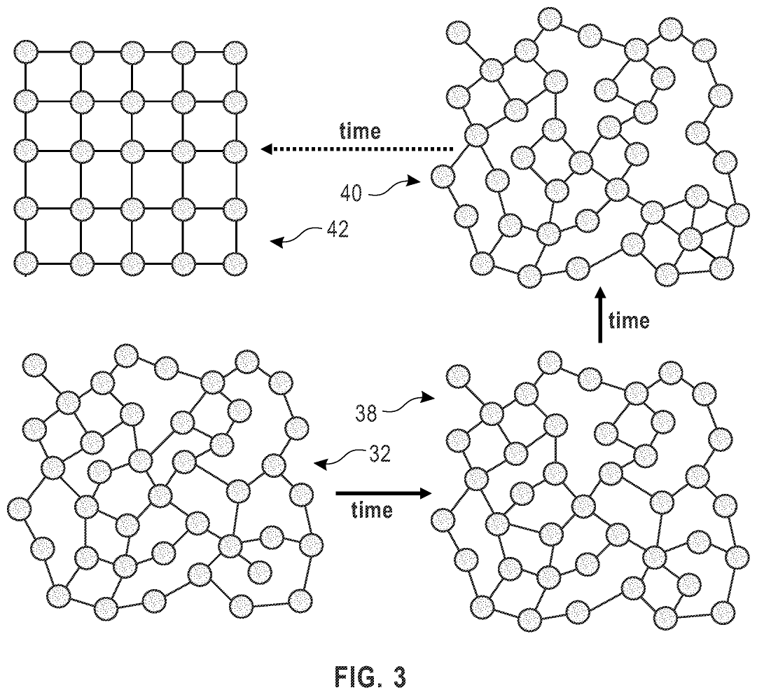

As shown in FIG. 2, during quenching, amorphous silicon is produced from liquid silicon upon reducing the temperature from T=8000K to T=2000K at regular intervals, with jumps of 1000K. During quenching, atoms move continuously making and breaking bonds, as depicted by the structure 30 at T=8000K and the structure 32 at T=5000K. The temperature is rapidly decreased to “freeze” atomic positions and generate the amorphous phase. Amorphous silicon atoms are frozen in positions similar to that of the liquid phase. By applying the LOM algorithm shown in equation (1) at each temperature T, the LOM processor 14 finds two different locally ordered geometries first appearing at T=5000K. One geometry is shown within dashed box 34 and the other is shown within dashed box 36. FIG. 3 shows structure 38 at temperature T=4000K, structure 40 at temperature T=3000K and structure 42 at temperature T=2000K. The two different locally ordered geometries that first appeared at T=5000K in structure 32 remain in structures 38 and 40 and eventually form a large crystalline structure 42 that would have very poor amorphous quality.

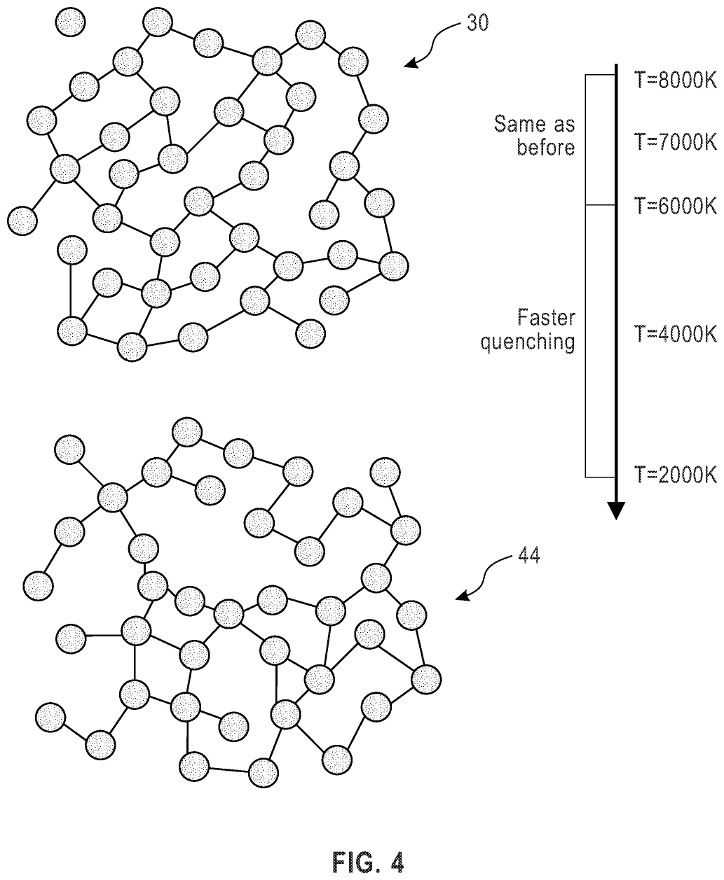

The LOM processor 14 is sensitive enough to look for and find small crystallites with different geometries that prior art simulation systems are not able to find. LOM processor 14 by showing the route by which the crystallites are produced, determines that they start forming at T=5000K. The presence of crystallites at T=5000K suggests a modification in the simulation protocol. Since the crystallites appear at T=5000K, a new simulation is generated using modified manufacturing conditions 28 that will speed temperature jumps from before 5000K down to lower temperatures. FIG. 4 shows the structures simulated under the modified condition having a more rapid temperature reduction jumping from 6000K to 4000K and jumping from 4000K to 2000K. The new simulation protocol produces crystallites-free amorphous structure at any temperature during quenching, resulting in a much higher quality amorphous silicon.

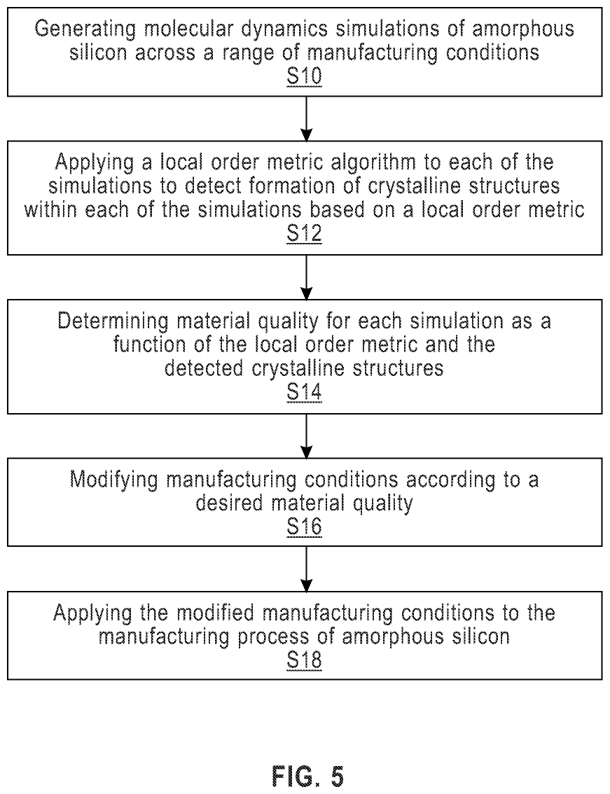

FIG. 5 is a flow diagram of one embodiment of a method for optimizing a process for the manufacture of amorphous silicon. The method as shown in FIG. 5 includes step S10 of generating molecular dynamics simulations of amorphous silicon across a range of manufacturing conditions. Step S12 includes applying a local order metric algorithm to each of the simulations to detect formation of crystalline structures within each of the simulations based on a local order metric determined by the algorithm. In one embodiment step S12 includes applying the local order metric algorithm at each simulated manufacturing condition. In one embodiment step S12 includes determining the manufacturing conditions at which the crystalline structures are formed. Step S14 includes determining material quality for each simulation as a function of the local order metric and the detected crystalline structures. Step S16 includes modifying manufacturing conditions according to a desired material quality. In one embodiment step S16 includes modifying the manufacturing conditions at which the crystalline structures are formed. Step S18 includes applying the modified manufacturing conditions to the manufacturing process of amorphous silicon.

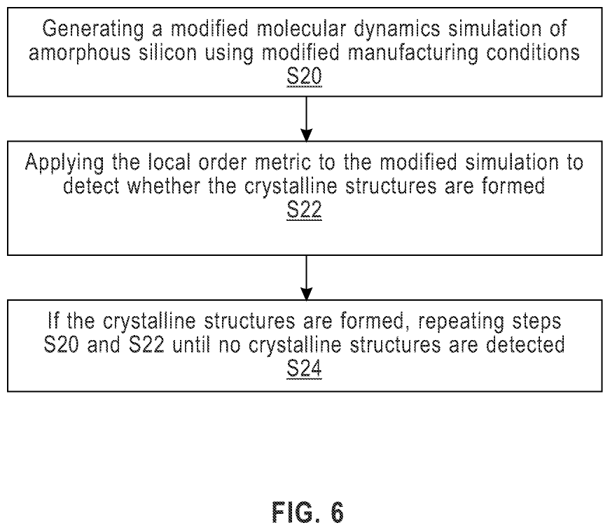

FIG. 6 is a flow diagram of one embodiment modifying manufacturing conditions according to a desired material quality, which includes step S20 of generating a modified molecular dynamics simulation of amorphous silicon using modified manufacturing conditions, step S22 of applying the local order metric to the modified simulation to detect whether the crystalline structures are formed and in step S24, if the crystalline structures are formed, repeating steps S20 and S22 until no crystalline structures are detected.

FIG. 7 illustrates a schematic of an example computer or processing system that may implement the method for optimizing a process for the manufacture of amorphous silicon in one embodiment of the present disclosure. The computer system is only one example of a suitable processing system and is not intended to suggest any limitation as to the scope of use or functionality of embodiments of the methodology described herein. The processing system shown may be operational with numerous other general purpose or special purpose computing system environments or configurations. Examples of well-known computing systems, environments, and/or configurations that may be suitable for use with the processing system shown in FIG. 7 may include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

The components of computer system may include, but are not limited to, one or more processors or processing units 100, a system memory 106, and a bus 104 that couples various system components including system memory 106 to processor 100. The processor 100 may include a program module 102 that performs the methods described herein. The module 102 may be programmed into the integrated circuits of the processor 100, or loaded from memory 106, storage device 108, or network 114 or combinations thereof. The processors 100 and program modules 102 can be programmed to perform the functions of the LOM 14 and material quality engine 20.

Bus 104 may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system may include a variety of computer system readable media. Such media may be any available media that is accessible by computer system, and it may include both volatile and non-volatile media, removable and non-removable media.

System memory 106 can include computer system readable media in the form of volatile memory, such as random access memory (RAM) and/or cache memory or others. Computer system may further include other removable/non-removable, volatile/non-volatile computer system storage media. By way of example only, storage system 108 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 104 by one or more data media interfaces.

Computer system may also communicate with one or more external devices 116 such as a keyboard, a pointing device, a display 118, etc.; one or more devices that enable a user to interact with computer system; and/or any devices (e.g., network card, modem, etc.) that enable computer system to communicate with one or more other computing devices. Such communication can occur via Input/Output (I/O) interfaces 110.

Still yet, computer system can communicate with one or more networks 114 such as a local area network (LAN), a general wide area network (WAN), and/or a public network (e.g., the Internet) via network adapter 112. As depicted, network adapter 112 communicates with the other components of computer system via bus 104. It should be understood that although not shown, other hardware and/or software components could be used in conjunction with computer system. Examples include, but are not limited to: microcode, device drivers, redundant processing units, external disk drive arrays, RAID systems, tape drives, and data archival storage systems, etc.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

In addition, while preferred embodiments of the present invention have been described using specific terms, such description is for illustrative purposes only, and it is to be understood that changes and variations may be made without departing from the spirit or scope of the following claims.

What is claimed is:

1. A computer implemented method for for optimizing a process for the manufacture of amorphous silicon, comprising:
generating molecular dynamics simulations of amorphous silicon across a range of manufacturing conditions;
applying a local order metric algorithm to each of the simulations to detect formation of crystalline structures within each of the simulations based on a local order metric determined by the algorithm;
determining material quality for each simulation as a function of the local order metric and the detected crystalline structures;
modifying manufacturing conditions according to a desired material quality; and
applying the modified manufacturing conditions to the manufacturing process of amorphous silicon.

2. The method of claim 1, further comprising applying the local order metric algorithm at each simulated manufacturing condition.

3. The method of claim 2, further comprising determining the manufacturing conditions at which the crystalline structures are formed.

4. The method of claim 3, further comprising modifying the manufacturing conditions at which the crystalline structures are formed.

5. The method of claim 4, further comprising:
(a) generating a modified molecular dynamics simulation of amorphous silicon using the modified manufacturing conditions;
(b) applying the local order metric to the modified simulation to detect whether the crystalline structures are formed;
(c) if the crystalline structures are formed, repeating steps (a) and (b) until no crystalline structures are detected.

6. The method of claim 3, wherein the manufacturing conditions at which the crystalline structures are formed is determined based on whether the local order metric is above a threshold value.

7. The method of claim 6, further comprising determining whether the local order metric of each atom of the simulated amorphous silicon is above the threshold value.

8. The method of claim 7, wherein, determining material quality is based on the number of atoms having the local order metric above the threshold.

9. A computer system for optimizing a process for the manufacture of amorphous silicon, comprising:
one or more computer processors;
one or more non-transitory computer-readable storage media;
program instructions, stored on the one or more non-transitory computer-readable storage media, which when implemented by the one or more processors, cause the computer system to perform the steps of:
generating molecular dynamics simulations of amorphous silicon across a range of manufacturing conditions;
applying a local order metric algorithm to each of the simulations to detect formation of crystalline structures within each of the simulations based on a local order metric determined by the algorithm;
determining material quality for each simulation as a function of the local order metric and the detected crystalline structures;
modifying manufacturing conditions according to a desired material quality; and
applying the modified manufacturing conditions to the manufacturing process of amorphous silicon.

10. The computer system of claim 9, further comprising applying the local order metric algorithm at each simulated manufacturing condition.

11. The computer system of claim 9, further comprising determining the manufacturing conditions at which the crystalline structures are formed and modifying the manufacturing conditions at which the crystalline structures are formed.

12. The computer system of claim 11, further comprising:
(a) generating a modified molecular dynamics simulation of amorphous silicon using the modified manufacturing conditions;
(b) applying the local order metric to the modified simulation to detect whether the crystalline structures are formed;
(c) if the crystalline structures are formed, repeating steps (a) and (b) until no crystalline structures are detected.

13. The computer system of claim 11, wherein the manufacturing conditions at which the crystalline structures are formed is determined based on whether the local order metric is above a threshold value and determining whether the local order metric of each atom of the simulated amorphous silicon is above the threshold value.

14. The computer of claim 13, wherein, determining material quality is based on the number of atoms having the local order metric above the threshold.

15. A computer program product comprising:
program instructions on a computer-readable storage medium, where execution of the program instructions using a computer causes the computer to perform a method for optimizing a process for the manufacture of amorphous silicon, comprising:
generating molecular dynamics simulations of amorphous silicon across a range of manufacturing conditions;
applying a local order metric algorithm to each of the simulations to detect formation of crystalline structures within each of the simulations based on a local order metric determined by the algorithm;
determining material quality for each simulation as a function of the local order metric and the detected crystalline structures;
modifying manufacturing conditions according to a desired material quality; and
applying the modified manufacturing conditions to the manufacturing process of amorphous silicon.

16. The computer program product of claim 15, further comprising applying the local order metric algorithm at each simulated manufacturing condition.

17. The computer program product of claim 16, further comprising determining the manufacturing conditions at which the crystalline structures are formed and modifying the manufacturing conditions at which the crystalline structures are formed.

18. The computer program product of claim 17, further comprising:
(d) generating a modified molecular dynamics simulation of amorphous silicon using the modified manufacturing conditions;
(e) applying the local order metric to the modified simulation to detect whether the crystalline structures are formed;
(f) if the crystalline structures are formed, repeating steps (a) and (b) until no crystalline structures are detected.

19. The method of claim 18, wherein the manufacturing conditions at which the crystalline structures are formed is determined based on whether the local order metric is above a threshold value and determining whether the local order metric of each atom of the simulated amorphous silicon is above the threshold value.

20. The method of claim 19, wherein, determining material quality is based on the number of atoms having the local order metric above the threshold.
